(19) United States
(12) Patent Application Publication
(10) Pub. No.: US 2014/0315274 A1
(43) Pub. Date: Oct. 23, 2014

(54) Methods For Production of Archeae Protease in Yeast

(71) Applicant: NOVOZYMES A/S, Bagsvaerd (DK)

(72) Inventor: Tomoka Matsui, Chiba (JP)

(73) Assignee: NOVOZYMES A/S, Bagsvaerd (DK)

(21) Appl. No.: 14/356,776

(22) PCT Filed: Nov. 5, 2012
(86) PCT No.: PCT/EP2012/071805

§ 371 Date: May 7, 2014

Related U.S. Application Data
(60) Provisional application No. 61/558,566, filed on Nov. 11, 2011.

(51) Int. Cl. C12N 9/60

(52) U.S. Cl. CPC C12N 9/60
USPC 435/224; 435/254.2; 435/254.21; 435/320.1

(57) ABSTRACT

The invention relates to: a method for producing a protease in yeast, comprising providing a yeast host cell comprising at least one polynucleotide expression construct encoding a first polypeptide secreted by the host cell in translational fusion with a second polypeptide, wherein the second polypeptide is a protease having a mature amino acid sequence similar to that of SEQ ID NO:2; the corresponding yeast host cell and the expression construct.

### REFERENCE TO SEQUENCE LISTING

This application contains a Sequence Listing in computer readable form. The computer readable form is incorporated herein by reference.

### FIELD OF INVENTION

The present invention relates to microbial production of a protease polypeptide in yeast by means of the expression of a fusion protein. The production exemplified below is of a protease originating from the Archeae Pyrococcus furiosus.

### BACKGROUND OF THE INVENTION

Proteins or polypeptides are currently being produced industrially in a number of ways, all involving cultivation of a microorganism which produces the protein. Several methods for optimizing industrial protein yield are known in the art and are being utilized routinely such as manipulations of the growth media, alterations of the microorganism e.g. by mutations, variations of the genetic elements like promoters and signal sequences etc. that influence expression of the gene encoding the desired protein, and also manipulations of the gene itself in order to e.g. enhance protein stability or activity.

Fusion-proteins have previously been described as a way of producing proteins that are otherwise difficult to obtain, one example could be active Human antibodies (Goshorn, S. C. et al., Cancer Research, 1993, Vol. 53(9) pp. 2123-2127) or production of industrial enzymes in a Bacillus host (WO 00/75344).

### SUMMARY OF THE INVENTION

Enzymes from extremophiles often have properties that are interesting for industrial applications, for example, a very high heat stability. However, they can be difficult to produce in quantities of industrial relevance and in active form.

The inventors found that a protease derived from the Archeae Pyrococcus furiosus was difficult to express in baker's yeast, Saccharomyces cerevisiae, even from a synthetic gene designed for expression in yeast together with a secretion signal, which normally works well in yeast (see Example 1).

However, it was surprisingly found, that the Archeae protease could be produced and secreted by Saccharomyces cerevisiae in its mature and active form, when it was expressed in translational fusion with either of three different heterologous fungal secreted carrier proteins (see Example 2).

Accordingly, in a first aspect, the invention relates to a method for producing a protease in yeast, comprising:
(a) providing a yeast host cell comprising at least one polynucleotide expression construct encoding a first polypeptide secreted by the host cell in translational fusion with a second polypeptide, wherein the second polypeptide is selected from the group consisting of:
   (i) a protease the mature part of which has an amino sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2; preferably to positions [1-412] (both included) of SEQ ID NO:2;
   (ii) a protease the mature part of which is encoded by a polynucleotide having a DNA sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1; preferably to positions [397-1632] (both included) of SEQ ID NO:1;
   (iii) a protease the mature part of which is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1; preferably with positions [397-1632] (both included) of SEQ ID NO:1; or the full-length complement of either;
   (iv) a variant of the mature polypeptide of SEQ ID NO: 2; preferably of positions [1-412] (both included) of SEQ ID NO:2, comprising a substitution, deletion, and/or insertion at one or more positions; and
   (v) a fragment of the polypeptide of (i), (ii), (iii), or (iv) that has protease activity
(b) cultivating the host cell under conditions conducive for the expression and secretion of the first polypeptide, whereby the translational fusion polypeptide is secreted and the mature protase is produced; and
(c) optionally, recovering the protease.

In a second aspect, the invention relates to a yeast host cell as defined in step (a) of the first aspect.

In a final aspect, the invention relates to a polynucleotide expression construct as defined in step (a) of the first aspect.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 412 of SEQ ID NO: 2.

Mature polypeptide coding sequence: The term “mature polypeptide coding sequence” means a polynucleotide that encodes a mature polypeptide having enzyme activity. In one aspect, the mature polypeptide coding sequence is nucleotides 397 to 1632 of SEQ ID NO: 1.

Fragment: The term “fragment” means a polypeptide having one or more (e.g., several) amino acids absent from the amino and/or carboxyl terminus of a mature polypeptide; wherein the fragment has retained its activity, for example a protease fragment which has kept the protease activity of the full-length polypeptide from which is was derived.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and either 35% formamide, following standard Southern blotting procedures for 12 to 24 hours. The carrier material is finally washed three times each for 15 minutes using 2×SSC, 0.2% SDS at 60° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having retained its activity.

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

### Methods of Production

The first aspect of the present invention relates to methods for producing a protease in yeast, comprising:
(a) providing a yeast host cell comprising at least one polynucleotide expression construct encoding a first polypeptide secreted by the host cell in translational fusion with a second polypeptide, wherein the second polypeptide is selected from the group consisting of:
   (i) a protease the mature part of which has an amino sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2; preferably to positions [1-412] (both included) of SEQ ID NO:2;
   (ii) a protease the mature part of which is encoded by a polynucleotide having a DNA sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1; preferably to positions [397-1632] (both included) of SEQ ID NO:1;
   (iii) a protease the mature part of which is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1; preferably with positions [397-1632] (both included) of SEQ ID NO:1; or the full-length complement of either;
   (iv) a variant of the mature polypeptide of SEQ ID NO: 2; preferably of positions [1-412] (both included) of SEQ ID NO:2, comprising a substitution, deletion, and/or insertion at one or more positions; and
   (v) a fragment of the polypeptide of (i), (ii), (iii), or (iv) that has protease activity
(b) cultivating the host cell under conditions conducive for the expression and secretion of the first polypeptide, whereby the translational fusion polypeptide is secreted and the mature protase is produced; and
(c) optionally, recovering the protease.

The host cells are cultivated in a nutrient medium suitable for production of the polypeptide using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The protease polypeptide may be detected using methods known in the art that are specific for the protease polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Variants

The first aspect of the present invention also relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### Translational Fusion

We mentioned in the background section, that a hard-to-express enzyme was reportedly successfully produced in Bacillus in translational fusion with a highly expressed and secreted native protein, a pectate lyase, which was fused at its C-terminal to the enzyme in order to have the native protein serve as a carrier or tractor (WO 00/75344).

Similarly, the protease polypeptide of the present invention is expressed as a fusion polypeptide in which a secreted polypeptide is fused to the N-terminus of the protease of the present invention.

A fusion polypeptide is produced by fusing a polynucleotide encoding another polypeptide to a polynucleotide encoding the protease of the present invention. Techniques for producing fusion polypeptides are known in the art, and include ligating the coding sequences encoding the polypeptides so that they are in frame and that expression of the fusion polypeptide is under control of the same promoter(s) and terminator.

It may be advantageous to include a small peptide linker between the fused polypeptides in order to physically allow them to fold correctly and to enable easy, perhaps even spontaneous, cleavage of the fusion polypeptide after its secretion to release the protease of the invention.

If the fusion polypeptide does not mature on its own accord after secretion to release the mature protease, it may further be engineered to comprise a specific proteolytic cleavage site between the two polypeptides in translational fusion. Upon secretion of the fusion protein, the site is then cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

In a preferred embodiment, the at least one polynucleotide expression construct further comprises a third polynucleotide encoding a linker peptide in translational fusion with and situated between the first polypeptide and the second polypeptide; preferably the third polynucleotide encodes a linker peptide; preferably the linker peptide comprises 2-200 amino acids; preferably 3-100 amino acids, e.g. 4-50 or 5-25 amino acids; and most preferably the linker peptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 11.

In a preferred embodiment, the first polypeptide of the translational fusion is a full-length or C-terminally truncated native or heterologous native or heterologous enzyme; preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

In a preferred embodiment, as exemplified below, the first polypeptide of the translational fusion is a full-length or C-terminally truncated native or heterologous glucoamylase; preferably a full-length or C-terminally truncated native or heterologous glucoamylase polypeptide selected from the group consisting of:

(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;

(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 5, (ii) the AMG gene inserted in the plasmid AMG 1 in the E. coli strain deposited with the Deutsche Sammlung von Mikroorganismen und Zellkulturen GmbH (DSMZ) on Nov. 23, 2009, and assigned accession number DSM 23123, or (iii) the full-length complement of (i) or (ii); and

(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 5 or the mature polypeptide coding sequence of the AMG gene inserted in the plasmid AMG 1 in the E. coli strain deposited with the Deutsche Sammlung von Mikroorganismen und Zellkulturen GmbH (DSMZ) on Nov. 23, 2009, and assigned accession number DSM 23123.

In another preferred embodiment, as exemplified below, the first polypeptide of the translational fusion is a full-length or C-terminally truncated native or heterologous phytase; preferably a full-length or C-terminally truncated native or heterologous phytase polypeptide selected from the group consisting of:

(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8;

(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 7 or the full-length complement of thereof; and

(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7.

In yet another preferred embodiment, as exemplified below, the first polypeptide of the translational fusion is a full-length or C-terminally truncated native or heterologous alpha-amylase; preferably a full-length or C-terminally truncated native or heterologous alpha-amylase polypeptide selected from the group consisting of:

(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;

(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 9 or the full-length complement of thereof; and

(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 9.

### Sources of Polypeptides Having Protease Activity

A polypeptide having protease activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The protease polypeptide may be an Archeae polypeptide. For example, the polypeptide may be of the Thermococci class, such as from the order of Thermococcales, from the family of Thermococcaceae or even from the genus Pyrococcus. In one aspect, the protease polypeptide may be a Pyrococcus furiosus polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a protease polypeptide of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Pyrococcus, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence in positions 397-1632 of SEQ ID NO: 1, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The third aspect of the invention relates to polynucleotide expression constructs as defined in step (a) of the first aspect.

The present invention also relates to nucleic acid constructs comprising a polynucleotide of the present invention operably linked to one or more control sequences that direct the expression of the coding sequence in a suitable host cell under conditions compatible with the control sequences.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated native or heterologous, and hybrid promoters, and may be obtained from genes encoding extracellular or intracellular polypeptides either homologous or heterologous to the host cell.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound.

Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The second aspect of the invention relates to yeast host cells as defined in step (a) of the first aspect.

Preferably, the yeast host cells of the second aspect comprise at least one expression polynucleotide construct of the present invention, wherein the protease-encoding gene is operably linked to one or more control sequences that direct the production of the fusion polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication.

The host cell is a yeast cell. The term “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

Fungal cells may be transformed by a process involving protoplast formation, transformation of the protoplasts, and regeneration of the cell wall in a manner known per se. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### EXAMPLES

### Materials and Methods

### Strains:

Saccharomyces cerevisiae YNG318: MATa Dpep4[cir+] ura3-52, leu2-D2, his 4-539 is described in J. Biol. Chem. 272 (15), 9720-9727, 1997.

Escherichia coli DH12S (available from Gibco BRL) is used for yeast plasmid rescue.

### Plasmids:

All yeast expression vectors are S. cerevisiae and E. coli shuttle vectors under the control of TPI promoter, constructed from pJC039 described in WO200192502.

### Genes:

A synthetic version of the Archeae Pyrococcus furiosus serine protease gene is shown in SEQ ID NO:1. The encoded mature amino acid sequence is shown in SEQ ID NO:2 (GeneSeqP:AAW2412), where the first 23 amino acids constitute the secretion signal peptide, the next 109 aa an N-terminal propeptide and the final 110 aa is a C-terminal pro-peptide; both pro-peptides are cleaved from the expressed polypeptide on maturation into its active form.

A polynucleotide sequence encoding a signal sequence from Humicola insolens is shown in SEQ ID NO:3, the signal peptide is shown in SEQ ID NO:4 (GeneSeqP:AEE85970).

The glucoamylase gene from Penicillium oxalicum was cloned into the pGEM-T vector (Promega Corporation, Madison, Wis., USA) using a pGEM-T Vector System (Promega Corporation, Madison, Wis., USA) to generate plasmid AMG 1. The sequence of the AMG gene inserted in the plasmid AMG 1 was confirmed by sequencing. The corresponding cDNA sequence of the glucoamylase gene is shown in SEQ ID NO: 5 and the amino acid of the encoded enzyme in SEQ ID NO:6.

The phytase gene from Peniophora lycii CBS 686.96 encodes a phytase having the amino acid sequence of GeneSeqP:AAW62858; the corresponding wildtype cDNA sequence is shown in SEQ ID NO: 7 with the aa-sequence shown in SEQ ID NO:8.

The cDNA encoding an acid alpha-amylase from Aspergillus niger is shown in SEQ ID NO:9, the encoded aa-sequence is shown in SEQ ID NO:10 (UNIPROT:P56271).

A linker region originally isolated from Athelia rolfsii is shown in SEQ ID NO:11, the encoded aa-sequence is shown in SEQ ID NO:12.

### Media and Substrates:

YPD: 20 g/L Glucose, 20 g/L Pepton and 10 g/L Yeast extract.
10× Basal solution: 66.8 g/l Yeast nitrogen base w/o amino acids (DIFCO), 100 g/l succinate and 60 g/l NaOH.
SC-glucose (or SC-medium): 100 ml/l 20% glucose (i.e., a final concentration of 2%=2 g/100 ml), 4 ml/l 5% threonine, 10 ml/l 1% tryptophan, 25 ml/l 20% casamino acids and 100 ml/l 10× basal solution. This solution was sterilized using a filter of a pore size of 0.20 Microm. Agar and H₂O (approx. 761 ml) is autoclaved together, and the separately sterilized SC-glucose solution is added to the agar solution.
PEG/LiAc solution: 50 ml 40% PEG4000 (sterilized by autoclaving) and 1 ml 5M Lithium Acetate (sterilized by autoclaving).
YPD: Bacto peptone 20 g/l, yeast extract 10 g/l, 20% glucose 100 ml/l.
Zein agar plate: 0.05-0.1% of zein (Sigma) and 2% of agar at 20 mM sodium acetate buffer, pH 6.

### DNA Manipulations:

Unless otherwise stated, DNA manipulations and transformations were performed using standard methods of molecular biology as described in Sambrook et al. (1989) Molecular cloning: A laboratory manual, Cold Spring Harbor lab. Cold Spring Harbor, N.Y.; Ausubel, F. M. et al. (eds.) “Current protocols in Molecular Biology”, John Wiley and Sons, 1995; Harwood, C. R. and Cutting, S. M. (eds.).

### Yeast Transformation:

To transform yeast, the in vivo recombinant mechanism was utilised by which it is possible for yeast to recombine a vector sequence and PCR fragments in vivo to create an expression vector, if both the vector sequence and the PCR fragments have the same flanking regions.

A DNA mixture was prepared by mixing 0.5 microliter of vector (HindIII-XbaI digested) and 1 microliter of PCR fragments. S. cerevisiae YNG318 competent cells were thawed on ice. 100 microliter of the cells were mixed with the DNA mixture and 10 microliter of carrier DNA in 12 ml polypropylene tubes. 0.6 ml PEG/LiAc solution was added and mixed gently and then incubated for 30 min at 30° C., and 200 rpm. Thereafter it was incubated for 30 min at 42° C. (heat shock) before transferring it to an eppendorf tube and centrifuging for 5 sec. The supernatant was removed and resolved in 3 ml of YPD. The cell suspension was then incubated for 45 min at 200 rpm at 30° C. before it was poured on SC-glucose plates.

### PCR Reaction:

Unless otherwise indicated the PCR reactions were carried out as follows. The PCR reaction contained 48.5 microliter water, 0.5 microliter of 100 pmol/microliter primers, 0.5 microliter template DNA and 2 beads of PuReTaq Ready-To-Go™ PCR Beads (GE Healthcare) was carried out under the following conditions:

| | Temp | |
|---|---|---|
| | (° C.) | Time |
| 1 | 94 | 2:00 |
| 2 | 94 | 0:30 |
| 3 | 55 | 0:30 |
| 4 | 72 | 1:30 |
| | go to 2 | 25 times |
| 5 | 72 | 10:00 |

DNA fragments were recovered from an agarose gel by the Qiagen gel extraction Kit. The resulting purified fragments were mixed with the vector digest. The mixed solution was used for Sacharomyces cerevisiae transformation via in vivo recombination.

### Other Methods:

E. coli transformation to rescue yeast plasmids was carried out by electroporation (BIO-RAD™ Gene Pulser).

Plasmid DNA was prepared with the Qiagen® Plasmid Kit. DNA fragments and recovered from agarose gel by the Qiagen® gel extraction Kit.

PCR was carried out by the PTC-200 DNA Engine.

The ABI PRISM™ 310 Genetic Analyzer was used for determination of all DNA sequences.

Yeast plasmid was extracted by Zymoprep Yeast Plasmid Minipreparation Kit (Zymo Research).

Yeast transformants were cultivated in 24 well-micro titer plates or shake flasks containing YPD.

### Protease Azo-Casein Assay:

20 microL of samples were mixed with 150 microL of substrate solution (4 ml of 12.5% azo-casein in ethanol in 96 ml of 20 mM sodium acetate, pH 4.5, containing 0.01% triton-100) and incubated for 4 hours or longer. After adding 20 microL/well of 100% trichloroacetic acid (TCA) solution, the plate was centrifuge and 100 microL of supernatants were pipette out to measure A₄₄₀.

### Zein Plate Assay:

Culture supernatants were spotted on zein agar plates (0.1% zein, 100 mM sodium acetate buffer, pH4) and incubated at 70° C. overnight to see clearing zone by enzymatic reaction.

### Phytase Plate Assay:

Culture supernatants were spotted on phytate agar plates (0.1% sodium phytate, 100 mM sodium acetate buffer, pH4) and incubated at 37° C. overnight. 0.5M CaCl₂ solution was poured onto the plate to precipitate calcium phytate o see clearing zone by enzymatic reaction.

### Glucoamylase and Alpha-Amylase Plate Assay:

Culture supernatants were spotted on starch agar plates (0.5% starch, 100 mM sodium acetate buffer, pH4) and incubated at 37° C. overnight followed by iodine staining to see clearing zones by enzymatic reaction.

### Example 1

### Expression of an Archeae Serine Protease in Saccharomyces cerevisiae

The synthetic gene of SEQ ID NO:1, encoding the Archeae Pyrococcus protease of SEQ ID NO:2, was amplified with the primer pairs, PfuF (SEQ ID NO: 13) and PfuR (SEQ ID NO:14), as well as Cuti-pfuF (SEQ ID NO:15) and PfuR (SEQ ID NO:14), where the former pair was used to amplify the native construct with its own signal sequence and the latter pair was used to construct a nucleic acid sequence (denoted cuti-PfuS) encoding the signal sequence from H. insolens cutinase (SEQ ID NO:4) in fusion with the N-terminal pro-mature-pro protease gene from Pyrococcus furiosus.

Yeast was transformed by introducing the resulting PCR fragments into S. cerevisiae YNG318 together with the pJCO39 vector digested with HindIII and XbaI, and PvuII and XbaI, respectively. The obtained transformants were cultivated in SC-glucose agar containing 0.1% zein. The results are shown in table 1; the cuti-PfuS transformants showed a faint halo around a colony due to protease activity. No halo was observed with pre-PfuS.

| | |
|---|---|
| PfuF (SEQ ID NO: 13): | aacgacggtacccggggat |
| | caagcttatgaaaggcctc |
| | aaggcattg |
| PfuR (SEQ ID NO: 14): | ctaattacatgatgcggcc |
| | ctctagattaggacgaacc |
| | aggctgc |
| cuti-pfuF (SEQ ID NO: 15): | gccttgttgctgctctcccc |
| | gcgcctgagaaaaaggtgg |

TABLE 1

| | Activity on zein plate |
|---|---|
| Pyrococcus protease with H. insolens | (+) |
| cutinase signal peptide | |
| Pyrococcus protease with | − |
| its own signal peptide | |

### Example 2

### Expression of the Pyrococcus Protease Gene as a Fusion Protein

Expression vectors were constructed for fusions of well-expressed enzyme proteins with the Archeae Pyrococcus protease to employ the well-expressed enzymes as a carrier or tractor proteins.

PCR reactions were carried out with the primer pairs, PhyF (SEQ ID NO:16) and PhyR (SEQ ID NO:17), AMG-F (SEQ ID NO:18) and AMG-R (SEQ ID NO:19), as well as amyF (SEQ ID NO:20) and amyR (SEQ ID NO:21), respectively, to amplify the Peniophora lycii phytase gene, the Penicillium oxalicum AMG gene and the Aspergillus niger acid alpha-amylase gene. The carrier enzyme genes and the protease gene were connected via the linker of SEQ ID NO: 11.

| | |
|---|---|
| PhyF (SEQ ID NO: 16): | aacgacggtacccggggatcaagc |
| | ttatggtttcttcggcattcg |
| PhyR (SEQ ID NO: 17): | tccacctttttctcaggcgcacta |
| | cccgaggagccacccgggcttgta |
| | gcaccttccgacggaacaaagc |
| AMG-F (SEQ ID NO: 18): | aacgacggtacccggggatcaagc |
| | ttatgcgtctcactctattatc |
| AMG-R (SEQ ID NO: 19): | tccacctttttctcaggcgcacta |
| | cccgaggagccacccgggcttgta |
| | gcaccacaagtggttgggactt |
| amyF (SEQ ID NO: 20): | aacgacggtacccggggatcaagc |
| | ttatgagattatcgacttcgag |
| amyR (SEQ ID NO: 21): | tccacctttttctcaggcgcacta |
| | cccgaggagccacccgggcttgta |
| | gcacctcttccgctcccgccac |

The resultant PCR fragments were mixed with the plasmid, cuti-PfuS, digested with HindIII, respectively and transformed into S. cerevisiae. The obtained transformants were cultivated in SC-glucose agar containing 0.1% zein. All fusion protease transformants showed much larger halos around the colonies than the construct with only the protease gene.

The transformants were also cultivated in 24 well plates containing 1 ml of YPD at 27° C. for 3 days and then the supernatants were tested for protease activity and also for the other enzyme activity as described in the Methods section.

These results indicate that the expression level in yeast of the Archeae Pyrococcus protease is much improved when the protease is expressed as a fusion protein.

TABLE 2
Symbol “−” means that no activity could be detected; “(+)” means only a
very faint activity could be observed and “++” means that enzyme activity
was clearly visible.

| | | | Carrier |
|---|---|---|---|
| Construct | Carrier enzyme | Protease activity | enzyme activity |
| pro-PfuS | — | − | − |
| cuti-PfuS | — | (+) | − |
| Phy-PfuS | phytase | ++ | ++ |
| AMG-PfuS | glucoamylase | ++ | ++ |
| Amy-PfuS | alpha-amylase | ++ | ++ |

1. A method for producing a protease in yeast, comprising:
(a) providing a yeast host cell comprising at least one polynucleotide expression construct encoding a first polypeptide secreted by the host cell in translational fusion with a second polypeptide, wherein the second polypeptide is selected from the group consisting of:
(i) a protease the mature part of which has an amino sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2; preferably to positions [1-412] of SEQ ID NO:2;
(ii) a protease the mature part of which is encoded by a polynucleotide having a DNA sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1; preferably to positions [397-1632] of SEQ ID NO:1;
(iii) a protease the mature part of which is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1; preferably with positions [397-1632] of SEQ ID NO:1; or the full-length complement of either;
(iv) a variant of the mature polypeptide of SEQ ID NO: 2; preferably of positions [1-412] of SEQ ID NO:2, comprising a substitution, deletion, and/or insertion at one or more positions; and
(v) a fragment of the polypeptide of (i), (ii), (iii), or (iv) that has protease activity
(b) cultivating the host cell under conditions conducive for the expression and secretion of the first polypeptide, whereby the translational fusion polypeptide is secreted and the mature protase is produced; and
(c) optionally, recovering the protease.

2. The method of claim 1, wherein the yeast is a Saccharomyces, preferably Saccharomyces cerevisiae.

3. The method of claim 1, wherein, the first polypeptide is a full-length or C-terminally truncated native or heterologous enzyme; preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

4. The method of claim 3, wherein the first polypeptide is a full-length or C-terminally truncated native or heterologous glucoamylase; preferably a full-length or C-terminally truncated native or heterologous glucoamylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 5, (ii) the AMG gene inserted in the plasmid AMG 1 in the E. coli strain deposited with the Deutsche Sammlung von Mikroorganismen und Zellkulturen GmbH (DSMZ) on Nov. 23, 2009, and assigned accession number DSM 23123, or (iii) the full-length complement of (i) or (ii); and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 5 or the mature polypeptide coding sequence of the AMG gene inserted in the plasmid AMG 1 in the E. coli strain deposited with the Deutsche Sammlung von Mikroorganismen und Zellkulturen GmbH (DSMZ) on Nov. 23, 2009, and assigned accession number DSM 23123.

5. The method of claim 3, wherein the first polypeptide is a full-length or C-terminally truncated native or heterologous phytase; preferably a full-length or C-terminally truncated native or heterologous phytase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 7 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7.

6. The method of claim 3, wherein the first polypeptide is a full-length or C-terminally truncated native or heterologous alpha-amylase; preferably a full-length or C-terminally truncated native or heterologous alpha-amylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 9 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 9.

7. The method of claim 1, wherein the at least one polynucleotide expression construct further comprises a third polynucleotide encoding a linker peptide in translational fusion with and situated between the first polypeptide and the second polypeptide.

8. The method of claim 7, wherein the third polynucleotide encodes a linker peptide; preferably the linker peptide comprises 2-200 amino acids; preferably 3-100 amino acids, e.g. 4-50 or 5-25 amino acids; and most preferably the linker peptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 11.

9. A yeast host cell comprising at least one polynucleotide expression construct encoding a first polypeptide secreted by the host cell in translational fusion with a second polypeptide, wherein the second polypeptide is selected from the group consisting of:
(i) a protease the mature part of which has an amino sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2;
(ii) a protease the mature part of which is encoded by a polynucleotide having a DNA sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1;
(iii) a protease the mature part of which is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1 or the full-length complement thereof;
(iv) a variant protease of the mature polypeptide of SEQ ID NO: 2 comprising a substitution, deletion, and/or insertion at one or more positions; and
(v) a fragment of the polypeptide of (i), (ii), (iii), or (iv) that has protease activity.

10. The yeast host cell of claim 9, which is a Saccharomyces, preferably Saccharomyces cerevisiae.

11. The yeast host cell of claim 9, wherein, the first polypeptide is a full-length or C-terminally truncated native or heterologous enzyme; preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

12. The yeast host cell of claim 11, wherein the first polypeptide is a full-length or C-terminally truncated native or heterologous glucoamylase; preferably a full-length or C-terminally truncated native or heterologous glucoamylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 5, (ii) the AMG gene inserted in the plasmid AMG 1 in the E. coli strain deposited with the Deutsche Sammlung von Mikroorganismen and Zellkulturen GmbH (DSMZ) on Nov. 23, 2009, and assigned accession number DSM 23123, or (iii) the full-length complement of (i) or (ii); and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 5 or the mature polypeptide coding sequence of the AMG gene inserted in the plasmid AMG 1 in the E. coli strain deposited with the Deutsche Sammlung von Mikroorganismen and Zellkulturen GmbH (DSMZ) on Nov. 23, 2009, and assigned accession number DSM 23123.

13. The yeast host cell of claim 11, wherein the first polypeptide is a full-length or C-terminally truncated native or heterologous phytase; preferably a full-length or C-terminally truncated native or heterologous phytase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 7 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7.

14. The yeast host cell of claim 11, wherein the first polypeptide is a full-length or C-terminally truncated native or heterologous alpha-amylase; preferably a full-length or C-terminally truncated native or heterologous alpha-amylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 9 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 9.

15. The yeast host cell of claim 1, wherein the at least one polynucleotide expression construct further comprises a third polynucleotide encoding a linker peptide in translational fusion with and situated between the first polypeptide and the second polypeptide.

16. The yeast host cell of claim 15, wherein the third polynucleotide encodes a linker peptide; preferably the linker peptide comprises 2-200 amino acids; preferably 3-100 amino acids, e.g. 4-50 or 5-25 amino acids; and most preferably the linker peptide comprises an amino acid sequence having at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to SEQ ID NO: 11.

17. A polynucleotide expression construct encoding a first polypeptide secreted by the host cell in translational fusion with a second polypeptide, wherein the second polypeptide is selected from the group consisting of:
(i) a protease the mature part of which has an amino sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2;
(ii) a protease the mature part of which is encoded by a polynucleotide having a DNA sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1;
(iii) a protease the mature part of which is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1 or the full-length complement thereof;
(iv) a variant protease of the mature polypeptide of SEQ ID NO: 2 comprising a substitution, deletion, and/or insertion at one or more positions; and
(v) a fragment of the polypeptide of (i), (ii), (iii), or (iv) that has protease activity.

18. The polynucleotide expression construct of claim 17, wherein, the first polypeptide is a full-length or C-terminally truncated native or heterologous enzyme; preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

19. The polynucleotide expression construct of claim 18, wherein the first polypeptide is a full-length or C-terminally truncated native or heterologous glucoamylase; preferably a full-length or C-terminally truncated native or heterologous glucoamylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 5, (ii) the AMG gene inserted in the plasmid AMG 1 in the E. coli strain deposited with the Deutsche Sammlung von Mikroorganismen and Zellkulturen GmbH (DSMZ) on Nov. 23, 2009, and assigned accession number DSM 23123, or (iii) the full-length complement of (i) or (ii); and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 5 or the mature polypeptide coding sequence of the AMG gene inserted in the plasmid AMG 1 in the E. coli strain deposited with the Deutsche Sammlung von Mikroorganismen and Zellkulturen GmbH (DSMZ) on Nov. 23, 2009, and assigned accession number DSM 23123.

20. The polynucleotide expression construct of claim 18, wherein the first polypeptide is a full-length or C-terminally truncated native or heterologous phytase; preferably a full-length or C-terminally truncated native or heterologous phytase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 7 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7.

21. The polynucleotide expression construct of claim 18, wherein the first polypeptide is a full-length or C-terminally truncated native or heterologous alpha-amylase; preferably a full-length or C-terminally truncated native or heterologous alpha-amylase polypeptide selected from the group consisting of:
(a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;
(b) a polypeptide encoded by a polynucleotide that hybridizes under low stringency conditions, medium stringency conditions, medium-high stringency conditions, high stringency conditions, or very high stringency conditions with (i) the mature polypeptide cDNA coding sequence of SEQ ID NO: 9 or the full-length complement of thereof; and
(c) a polypeptide encoded by a polynucleotide having at least 60%, e.g., at least 65%, at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 9.

22. The polynucleotide expression construct of claim 1, wherein the at least one polynucleotide expression construct further comprises a third polynucleotide encoding a linker peptide in translational fusion with and situated between the first polypeptide and the second polypeptide.

23. The polynucleotide expression construct of claim 22, wherein the third polynucleotide encodes a linker peptide; preferably the linker peptide comprises 2-200 amino acids; preferably 3-100 amino acids, e.g. 4-50 or 5-25 amino acids; and most preferably the linker peptide comprises an amino acid sequence having at least 70%, at least 75%, at least 80%, at least 85%,%, at least 90%, at least 91%, at least 92%, at least 93%, at least 94%, at least 95%, at least 96%, at least 97%, at least 98%, at least 99%, or 100% sequence identity to SEQ ID NO: 11.


SEQUENCE LISTING (21 sequences)

SEQ ID NO: 1 - DNA, 1962 bp, artificial sequence
  Synthetic coding sequence
atgaaggggctgaaagctctcatattagtgattttagttctaggtttggtagtagggagcgtagcggcagctccagagaagaaagttgaacaagtaagaaatgttgagaagaactatggtctgctaacgccaggactgttcagaaaaattcaaaaattgaatcctaacgaggaaatcagcacagtaattgtatttgaaaaccatagggaaaaagaaattgcagtaagagttcttgagttaatgggtgcaaaagttaggtatgtgtaccatattatacccgcaatagctgccgatcttaaggttagagacttactagtcatctcaggtttaacagggggtaaagctaagctttcaggtgttaggtttatccaggaagactacaaagttacagtttcagcagaattagaaggactggatgagtctgcagctcaagttatggcaacttacgtttggaacttgggatatgatggttctggaatcacaataggaataattgacactggaattgacgcttctcatccagatctccaaggaaaagtaattgggtgggtagattttgtcaatggtaggagttatccatacgatgaccatggacatggaactcatgtagcttcaatagcagctggtactggagcagcaagtaatggcaagtacaagggaatggctccaggagctaagctggcgggaattaaggttctaggtgccgatggttctggaagcatatctactataattaagggagttgagtgggccgttgataacaaagataagtacggaattaaggtcattaatctttctcttggttcaagccagagctcagatggtactgacgctctaagtcaggctgttaatgcagcgtgggatgctggattagttgttgtggttgccgctggaaacagtggacctaacaagtatacaatcggttctccagcagctgcaagcaaagttattacagttggagccgttgacaagtatgatgttataacaagcttctcaagcagagggccaactgcagacggcaggcttaagcctgaggttgttgctccaggaaactggataattgctgccagagcaagtggaactagcatgggtcaaccaattaatgactattacacagcagctcctgggacatcaatggcaactcctcacgtagctggtattgcagccctcttgctccaagcacacccgagctggactccagacaaagtaaaaacagccctcatagaaactgctgatatcgtaaagccagatgaaatagccgatatagcctacggtgcaggtagggttaatgcatacaaggctataaactacgataactatgcaaagctagtgttcactggatatgttgccaacaaaggcagccaaactcaccagttcgttattagcggagcttcgttcgtaactgccacattatactgggacaatgccaatagcgaccttgatctttacctctacgatcccaatggaaaccaggttgactactcttacaccgcctactatggattcgaaaaggttggttattacaacccaactgatggaacatggacaattaaggttgtaagctacagcggaagtgcaaactatcaagtagatgtggtaagtgatggttccctttcacagcctggaagttcaccatctccacaaccagaaccaacagtagacgcaaagacgttccaaggatccgatcactactactatgacaggagcgacacctttacaatgaccgttaactctggggctacaaagattactggagacctagtgtttgacacaagctaccatgatcttgacctttacctctacgatcctaaccagaagcttgtagatagatcggagagtcccaacagctacgaacacgtagaatacttaacccccgccccaggaacctggtacttcctagtatatgcctactacacttacggttgggcttactacgagctgacggctaaagtttattatggc

SEQ ID NO: 2 - protein, 654 aa, artificial sequence
  Synthetic Construct
MKGLKALILVILVLGLVVGSVAAAPEKKVEQVRNVEKNYGLLTPGLFRKIQKLNPNEEISTVIVFENHREKEIAVRVLELMGAKVRYVYHIIPAIAADLKVRDLLVISGLTGGKAKLSGVRFIQEDYKVTVSAELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSSPSPQPEPTVDAKTFQGSDHYYYDRSDTFTMTVNSGATKITGDLVFDTSYHDLDLYLYDPNQKLVDRSESPNSYEHVEYLTPAPGTWYFLVYAYYTYGWAYYELTAKVYYG

SEQ ID NO: 3 - DNA, 54 bp, Humicola insolens
  CDS (1)..(54): Signal peptide
atgaagttcttcaccaccatcctcagcaccgccagccttgttgctgctctcccc

SEQ ID NO: 4 - protein, 18 aa, Humicola insolens
MKFFTTILSTASLVAALP

SEQ ID NO: 5 - DNA, 1455 bp, Penicillium oxalicum
  CDS (1)..(1455)
  sig_peptide (1)..(63)
  mat_peptide (64)..(1455)
atgcgtctcactctattatcaggtgtagccggcgttctctgcgcaggacagctgacggcggcgcgtcctgatcccaagggtgggaatctgacgccgttcatccacaaagagggcgagcggtcgctccaaggcatcttggacaatctcggtgggcgaggtaagaaaacacccggcactgccgcagggttgtttattgccagtccaaacacagagaatccaaactattattatacatggactcgtgactcagctttgactgccaagtgcttgatcgacctgttcgaagactctcgggcaaagtttccaattgaccgcaaatacttggaaacaggaattcgggactacgtgtcgtcccaagcaatcctccagagtgtgtctaatccttctggaaccctgaaggatggctctggtctgggtgaacccaagtttgagattgacctgaatcccttttcgggtgcctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttaccaaggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcgatgccgtggttcctccctcctggggcgcaaagtcggcaaacaaagtcccaaccacttgt

SEQ ID NO: 6 - protein, 485 aa, Penicillium oxalicum
MRLTLLSGVAGVLCAGQLTAARPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTC

SEQ ID NO: 7 - DNA, 1317 bp, Peniophora lycii
  CDS (1)..(1317)
  sig_peptide (1)..(90)
  mat_peptide (91)..(1317)
atggtttcttcggcattcgcaccttccatcctacttagcttgatgtcgagtcttgctttgagcacgcagttcagctttgttgcggcgcagctacctatccccgcacaaaacacaagtaattgggggccttacgatcccttctttcccgtcgaaccgtatgcagctccgccggaagggtgcacagtgacacaggtcaacctgattcagaggcacggcgcgcgttggcccacatccggcgcgcggtcgcggcaggtcgccgccgtagcgaagatacaaatggcgcgaccattcacggatcccaagtatgagttcctcaacgacttcgtgtacaagttcggcgtcgccgatctgctaccgttcggggctaaccaatcgcaccaaaccggcaccgatatgtatacgcgctacagtacactatttgagggcggggatgtaccctttgtgcgcgcggctggtgaccaacgcgtcgttgactcctcgacgaactggacggcaggctttggcgatgcttctggcgagactgttctcccgacgctccaggttgtgcttcaagaagaggggaactgcacgctctgcaataatatgtgcccgaatgaagtggatggtgacgaatccacaacgtggctgggggtctttgcgccgaacatcaccgcgcgattgaacgctgctgcgccgagtgccaacctctcagacagcgacgcgctcactctcatggatatgtgcccgttcgacactctcagctccgggaacgccagccccttctgtgacctatttaccgcggaggagtatgtgtcgtacgagtactactatgacctcgacaagtactatggcacgggccccgggaacgctctcggtcctgtccagggcgtcggatacgtcaatgagctgcttgcacgcttgaccggccaagccgttcgagacgagacgcagacgaaccgcacgctcgacagcgaccctgcaacattcccgctgaaccgtacgttctacgccgacttctcgcatgataacaccatggtgcccatctttgcggcgctcgggctcttcaacgccaccgccctcgacccgctgaagcccgacgagaacaggttgtgggtggactctaagctggtaccgttctctggacatatgacggtcgagaagctggcatgttctgggaaggaggcggtcagggtgctcgtgaacgacgcggtgcagccgctggagttctgcggaggtgttgatggggtgtgcgagctttcggctttcgtagagagccagacgtatgcgcgggagaatgggcaaggcgacttcgccaagtgcggctttgttccgtcggaa

SEQ ID NO: 8 - protein, 439 aa, Peniophora lycii
MVSSAFAPSILLSLMSSLALSTQFSFVAAQLPIPAQNTSNWGPYDPFFPVEPYAAPPEGCTVTQVNLIQRHGARWPTSGARSRQVAAVAKIQMARPFTDPKYEFLNDFVYKFGVADLLPFGANQSHQTGTDMYTRYSTLFEGGDVPFVRAAGDQRVVDSSTNWTAGFGDASGETVLPTLQVVLQEEGNCTLCNNMCPNEVDGDESTTWLGVFAPNITARLNAAAPSANLSDSDALTLMDMCPFDTLSSGNASPFCDLFTAEEYVSYEYYYDLDKYYGTGPGNALGPVQGVGYVNELLARLTGQAVRDETQTNRTLDSDPATFPLNRTFYADFSHDNTMVPIFAALGLFNATALDPLKPDENRLWVDSKLVPFSGHMTVEKLACSGKEAVRVLVNDAVQPLEFCGGVDGVCELSAFVESQTYARENGQGDFAKCGFVPSE

SEQ ID NO: 9 - DNA, 1503 bp, Aspergillus niger
  CDS (1)..(1503)
  sig_peptide (1)..(72)
  mat_peptide (73)..(1503)
atgagattatcgacttcgagtctcttcctttccgtgtctctgctggggaagctggccctcgggctgtcggctgcagaatggcgcactcagtcgatttacttcctattgacggatcggttcggtaggacggacaattcgacgacagctacatgcgatacgggtgaccaaatctattgtggtggcagttggcaaggaatcatcaaccatctggattatatccagggcatgggattcacggccatctggatctcgcctatcactgaacagctgccccaggatactgctgatggtgaagcttaccatggatattggcagcagaagatatacgacgtgaactccaacttcggcactgcagatgacctcaagtccctctcagatgcgcttcatgcccgcggaatgtacctcatggtggacgtcgtccctaaccacatgggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatctactggcaactcctctacgccttcgaatcctccagcggcagcatcagcaatctctacaacatgatcaaatccgtcgcaagcgactgctccgatccgacactactcggcaacttcatcgaaaaccacgacaatccccgtttcgcctcctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcgaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaaga

SEQ ID NO: 10 - protein, 501 aa, Aspergillus niger
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGR

SEQ ID NO: 11 - DNA, 33 bp, Corticiium rolfsii
  CDS (1)..(33): Peptide linker
ggtgctacaagcccgggtggctcctcgggtagt

SEQ ID NO: 12 - protein, 11 aa, Corticiium rolfsii
GATSPGGSSGS

SEQ ID NO: 13 - DNA, 47 bp, artificial sequence
  Primer PfuF
aacgacggtacccggggatcaagcttatgaaaggcctcaaggcattg

SEQ ID NO: 14 - DNA, 45 bp, artificial sequence
  Primer PfuR
ctaattacatgatgcggccctctagattaggacgaaccaggctgc

SEQ ID NO: 15 - DNA, 39 bp, artificial sequence
  Primer cuti-pfuF
gccttgttgctgctctccccgcgcctgagaaaaaggtgg

SEQ ID NO: 16 - DNA, 45 bp, artificial sequence
  Primer phyF
aacgacggtacccggggatcaagcttatggtttcttcggcattcg

SEQ ID NO: 17 - DNA, 70 bp, artificial sequence
  Primer phyR
tccacctttttctcaggcgcactacccgaggagccacccgggcttgtagcaccttccgacggaacaaagc

SEQ ID NO: 18 - DNA, 46 bp, artificial sequence
  Primer AMG-F
aacgacggtacccggggatcaagcttatgcgtctcactctattatc

SEQ ID NO: 19 - DNA, 70 bp, artificial sequence
  Primer AMG-R
tccacctttttctcaggcgcactacccgaggagccacccgggcttgtagcaccacaagtggttgggactt

SEQ ID NO: 20 - DNA, 46 bp, artificial sequence
  Primer amyF
aacgacggtacccggggatcaagcttatgagattatcgacttcgag

SEQ ID NO: 21 - DNA, 70 bp, artificial sequence
  Primer amyR
tccacctttttctcaggcgcactacccgaggagccacccgggcttgtagcacctcttccgctcccgccac